(19) United States
(12) Patent Application Publication
(10) Pub. No.: US 2020/0005674 A1
(43) Pub. Date: Jan. 2, 2020

(54) METHOD AND APPARATUS FOR AUSCULTATION TRAINING

(71) Applicant: Paul Jacques Charles Lecat, Kent, OH (US)

(72) Inventor: Paul Jacques Charles Lecat, Kent, OH (US)

(21) Appl. No.: 16/551,162

(22) Filed: Aug. 26, 2019

Related U.S. Application Data
(63) Continuation-in-part of application No. 15/664,622, filed on Jul. 31, 2017, now Pat. No. 10,395,558.
Application No. 15/664,622 is a continuation-in-part of application No. 15/429,957, filed on Feb. 10, 2017, now Pat. No. 10,395,557.
(60) Provisional application No. 62/308,254, filed on Mar. 15, 2016.
(60) Provisional application No. 62/296,855, filed on Feb. 18, 2016.

(51) Int. Cl. G09B 23/28; G09B 5/04; H04R 1/10; H04R 3/00; A61B 7/04

(52) U.S. Cl. CPC G09B 23/28; G09B 5/04; H04R 1/1016; H04R 2460/11; H04R 1/1075; A61B 7/04; H04R 3/00

(57) ABSTRACT

This disclosure is of an auscultation training device that includes: a headpiece; at least one earpiece; tubing having a hollow interior, connected between the headpiece and the at least one earpiece, and capable of transmitting sound from the headpiece to the at least one earpiece; a speaker inserted into the hollow interior of the tubing through an insertion point that forms an airtight seal with the tubing, wherein the speaker does not fully obstruct the hollow interior of the tubing; a vent tube having a hollow interior, a first end secured to the tubing such that the tubing hollow interior and vent tube hollow interior are in fluid communication, a second end, and an opening; and a vent solenoid secured to the second end of the vent tube and capable of blocking and unblocking the vent tube opening.

This application is a continuation-in-part of U.S. patent application Ser. No. 15/664,622, filed Jul. 31, 2017, which is a continuation-in-part of U.S. patent application Ser. No. 15/429,957, filed Feb. 10, 2017, which claims priority to U.S. Provisional Patent Application No. 62/296,855, filed Feb. 18, 2016, and U.S. Provisional Patent Application No. 62/308,254, filed Mar. 15, 2016, the contents of which are incorporated herein by reference.

### TECHNICAL FIELD

The present invention relates to medical training devices for use in instructing medical students. More particularly, the present invention relates to an auscultation training device and a method of use therefor.

### BACKGROUND

Auscultation is the act of listening to sounds within the body as a method of diagnosis. A stethoscope is an example of an auscultation device that is used in the medical field to listen to internal sounds in the human body, such as heart sounds, breathing (breath sounds), intestinal noises, blood flow through the arteries and veins, and externally produced sounds, such as percussion. Acoustic stethoscopes operate on the transmission of sound from a headpiece, via air filled tubes, to the listener's ears. The headpiece may include a diaphragm that can be placed against a human body for sensing sound. These body sounds then cause the diaphragm to vibrate, in turn creating acoustic pressure waves that travel up the hollow tubes to the listener's ears.

Forming a diagnosis of a patient using an auscultation device such as a stethoscope requires training in proper placement of the headpiece, detecting bodily sounds, and identifying abnormalities as compared to normal body sounds. Often this type of training takes advantage of simulated, or standardized, patients such as mannequins with recorded sounds being presented to the student. Other times, real human patients presenting with certain known ailments are used to train students to detect and identify body sounds in a real-time “live” environment.

In situations where a student is being trained using simulated sounds and patients, it is beneficial to use a stethoscope training aid that can recreate these simulated sounds through the earpiece of the stethoscope so the student can experience these sounds in as close to a natural condition as possible. One such device is disclosed in U.S. Pat. No. 7,645,141 to Lecat, the entire disclosure of which is fully incorporated herein by reference.

In live environments, students typically use a standard practice stethoscope to detect and identify body sounds; however, when teaching in groups, this type of environment tends to result in differing exposure between students. For example, student A may hear a body sound that is slightly different than what student B hears based on differences in placement of the stethoscope headpiece to changes in the sounds the human body makes at different times. This problem is compounded as the size of the student group increases. What is needed is a tool that allows for a blending of the live and simulated environments wherein the experience can be shared among multiple students simultaneously in order to standardize the learning experience.

### SUMMARY

In accordance with one aspect of the present invention, an auscultation training device includes: a headpiece; at least one earpiece; tubing having a hollow interior, connected between the headpiece and the at least one earpiece, and capable of transmitting sound from the headpiece to the at least one earpiece; a speaker inserted into the hollow interior of the tubing through an insertion point that forms an airtight seal with the tubing, wherein the speaker does not fully obstruct the hollow interior of the tubing; a vent tube having a hollow interior, a first end secured to the tubing such that the tubing hollow interior and vent tube hollow interior are in fluid communication, a second end, and an opening; and a vent solenoid secured to the second end of the vent tube and capable of blocking and unblocking the vent tube opening.

In accordance with another aspect of the present invention, a method of auscultation training includes the steps of: (a) providing an auscultation training device including: a headpiece; at least one earpiece; tubing having a hollow interior, connected between the headpiece and the at least one earpiece, and capable of transmitting sound from the headpiece to the at least one earpiece; a speaker inserted into the hollow interior of the tubing through an insertion point that forms an airtight seal with the tubing, wherein the speaker does not fully obstruct the hollow interior of the tubing; a vent tube having a hollow interior, a first end secured to the tubing such that the tubing hollow interior and vent tube hollow interior are in fluid communication, a second end, and an opening; and a vent solenoid secured to the second end of the vent tube and capable of blocking and unblocking the vent tube opening; (b) activating the vent solenoid to unblock the vent tube opening and vent the vent tube hollow interior and tubing hollow interior, dampening any sounds transmitted from the headpiece by the tubing to the at least one earpiece; (c) playing a sound by the speaker; and (d) transmitting the sound played by the speaker by the tubing to the at least one earpiece.

In accordance with another aspect of the present invention, an auscultation training device includes: a headpiece; at least one earpiece; tubing having a hollow interior, connected between the headpiece and the at least one earpiece, and capable of transmitting sound from the headpiece to the at least one earpiece, wherein the tubing has a vent opening; a speaker inserted into the hollow interior of the tubing through an insertion point that forms an airtight seal with the tubing, wherein the speaker does not fully obstruct the hollow interior of the tubing; and a vent solenoid secured to the tubing and capable of blocking and unblocking the vent opening.

### BRIEF DESCRIPTION OF THE DRAWINGS

FIG. 1 is a diagram of an auscultation training device;

FIG. 2 is a diagram of an embodiment of the training environment;

FIG. 3 is a diagram of a second embodiment of the training environment;

FIG. 4 is a diagram of a third embodiment of the training environment;

FIG. 5 is a diagram of a separate embodiment of an auscultation training device;

FIG. 6 is a diagram of a separate embodiment of an auscultation training device;

FIG. 7 is a diagram of a separate embodiment of an auscultation training device;

FIG. 8 shows a partial cross-section view of the vent tube and vent solenoid in a de-energized position, according to one embodiment; and

FIG. 9 shows a partial cross-section view of the vent tube and vent solenoid in an energized position, according to one embodiment.

### DESCRIPTION

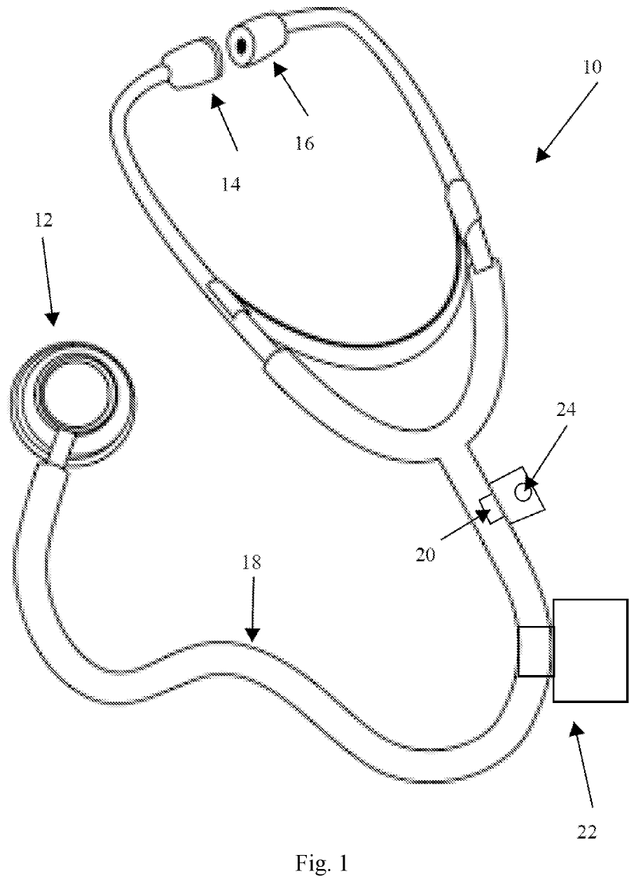

With reference to FIG. 1, an embodiment of an auscultation training device 10 is shown having a headpiece 12, which may be connected to a pair of earpieces 14, 16, and tubing 18, which can have a generally hollow interior. In some embodiments, the tubing 18 may be made in multiple parts, where one portion may run from the headpiece to just past the Y fork and be made from a more flexible material (e.g., plastic or rubber), and where the other portions may run from the earpieces to the first portions (just before the Y fork) and be made from a more rigid material (e.g., metal). The training device 10 can also have a speaker 20 that extends into the hollow interior of the tubing 18 without fully obstructing the hollow interior of the tubing 18. The insertion point of the speaker 20 can form an airtight seal with the tubing 18 as to allow normal and natural function of the stethoscope. The training device can also have a solenoid 22 inserted within the hollow interior of the tubing 18 between the headpiece 12 and the speaker 20. The insertion point of the solenoid 22 can also form an airtight seal with the tubing 18 as to allow normal and natural function of the stethoscope. The speaker 20 can be connected to an input source. In one embodiment, the speaker can be connected to a standard 3.5 mm audio jack 24 to allow the speaker 20 to be connected to an external audio output device, including simulated patients, training mannequins, body sound simulators, mp3 players, smartphones, tablets, CD players, DVD players, or any other device capable of transmitting audio signals through a standard 3.5 mm audio connection.

According to one embodiment, the training device 10 can be used as a standard stethoscope wherein a student places the headpiece 12 on a patient to receive body sounds through normal stethoscope operation. According to this embodiment, the speaker 20 and solenoid 22 are not activated and acoustic pressure waves are free to travel through the hollow interior of the tubing 18 to the earpieces 14, 16. This arrangement allows the student to operate the training device 10 in a manner that is consistent with a live diagnosis, thereby teaching the student proper placement of the headpiece 12 and encouraging development of the student's muscle memory to aid in learning and retention of proper placement techniques.

According to one embodiment, the speaker 20 can be connected to an external audio output device and activated in a manner that would allow the student to hear body sounds generated by either the placement of the headpiece 12, the speaker 20, or both. The external audio output device can be coordinated with the placement of the headpiece 12 such that a desired body sound can be broadcast simultaneously with the placement of the headpiece 12, thereby amplifying and enhancing the live sounds detected by the headpiece 12. Alternatively, the audio output device can play audio files through the speaker 20 at a separate time, thereby allowing the student to hear a live sound separate from a simulated sound for purposes of comparison and learning.

According to one embodiment, the solenoid 22 can be activated and can mechanically block the hollow interior of the tubing 18 between the headpiece 12 and the speaker 20. This mechanical block can function to dampen or silence the transmission of sound waves from the headpiece 12. The student can then exclusively use the external audio output device and speaker 20 to generate simulated sounds without interference from live sounds generated through the headpiece 12.

According to one embodiment, the solenoid 22 can be replaced with any physical means for temporarily or permanently blocking the hollow interior of the tubes 18 to allow exclusive use of the external audio output device and speaker 20 to generate simulated sounds without interference from live sounds generated through the headpiece 12.

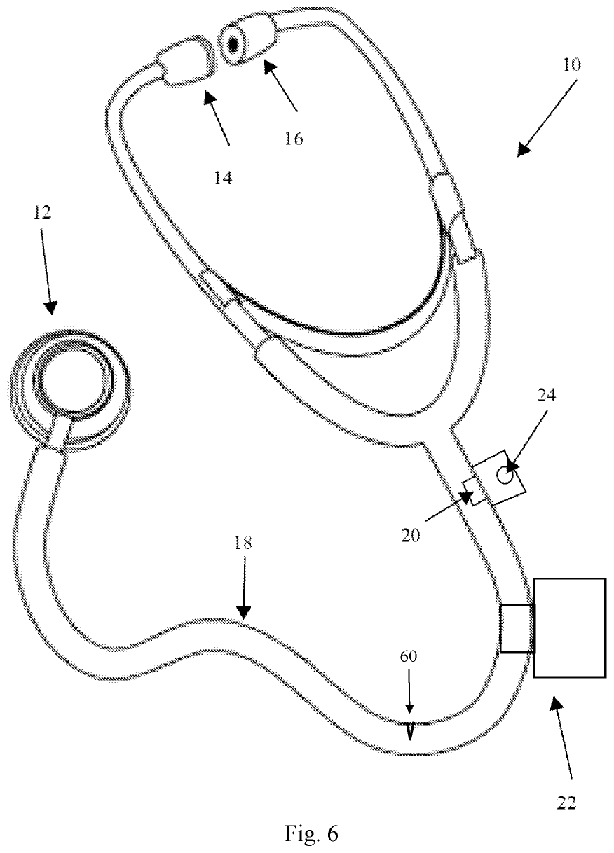

With reference to FIG. 6, in lieu of the solenoid, the wall of the tubing 18 can contain a small opening 60 or puncture to vent the tubing 18 and thereby dampen or lessen any live sounds generated through the headpiece 12. The opening 60 can be located below the speaker 20 which can allow the speaker 20 to function normally to transmit sounds. The presence of the opening 60 can disrupt or dampen the live sounds, but may not eliminate them completely. In some applications a user may find it useful to be able to faintly detect live sounds, for example, for comparison purposes with the recorded sounds. In another embodiment, the opening 60 can eliminate the live sounds completely.

According to one embodiment, the size and presence of the opening 60 can be controlled through an external control device (not shown in FIG. 6) that may allow a user to selectively open or close the opening 60, or to change the size of the opening 60. In some embodiments, a vent solenoid 64 (discussed below) may be the external control device. Further, the opening 60 can include a manual cap or cover which can function to cover the opening 60 and allow the transmission of live sounds according to the user's desired mode of operation.

According to another embodiment, the insertion point of the speaker 20 into the tubing 18 can intentionally be permitted to “leak,” i.e. not be air-tight. This “leaky” insertion can serve to vent the tubing similar to opening 60 which can cause live sounds from the headpiece 12 to be dampened or reduced.

According to one embodiment, the stethoscope 10 can include both the blocking solenoid 22 and opening 60. This embodiment may be useful in applications in which it might selectively be desirable to detect faint live sounds and in others it may be desirable to have live sounds completely blocked from reaching the earpieces 14, 16.

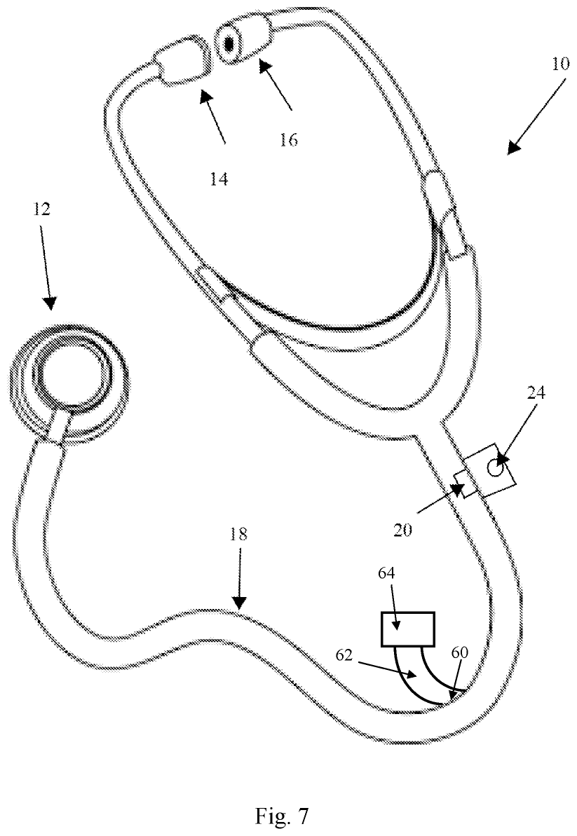

With reference to FIG. 7, according to another embodiment, one end of a vent tube 62 may be installed in the opening 60, with a vent solenoid 64 secured at the other end of the vent tube 62. The vent tube 62 may have a generally hollow interior, similar to the tubing 18, and the hollow interior of the vent tube 62 may be in fluid communication with the hollow interior of the tubing 18, such that air may travel between them. The vent solenoid 64 may be activated (whether by energizing or de-energizing, depending on the configuration) to unblock the opening 60 and may be de-activated to block the opening 60. In one embodiment, the vent solenoid 64 may be normally closed when de-energized, which may keep the vent tube 62 closed and thus keep the opening 60 closed also and allow any live sounds generated through the headpiece 12 to travel to the earpieces 14, 16. When the vent solenoid 64 is energized, it 64 may open the vent tube 62, thus venting the tubing 18 through the opening 60 and dampening or lessening any live sounds generated through the headpiece 12, as described above. In an alternative embodiment, the vent solenoid 64 may be normally open when de-energized with the vent tube 62 also open, and when the vent solenoid 64 is energized, it 64 may close, thus closing the vent tube 62.

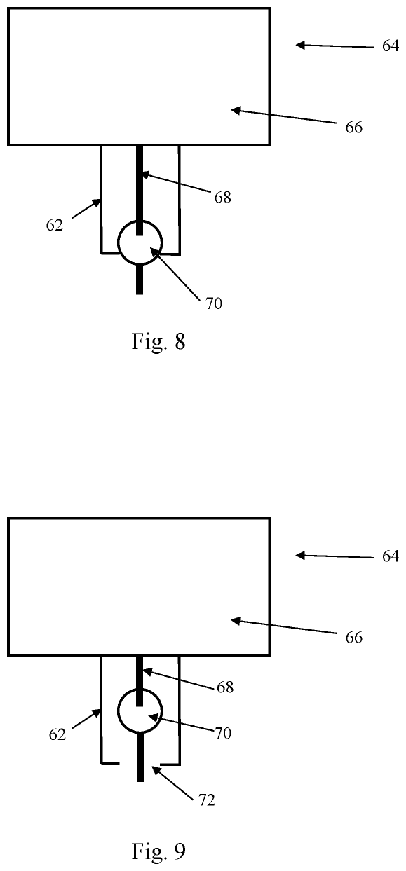

FIG. 8 shows a partial cross-sectional view of the vent tube 62 and vent solenoid 64 in the de-energized, normally closed position, according to one embodiment. With the solenoid coil 66 de-energized, the solenoid piston or plunger 68 may extend downwards by gravity out of the solenoid or its coil. The plunger 68 may have a plug 70 at the end or along the plunger 68. The plug 70 may be made of foam (including, but not limited to, closed-cell extruded polystyrene foam), cotton, rubber, paper fiber, plastic, cloth, leather, wool, or any other soft material, or of a magnetic material coated with soft material. When the vent solenoid 64 is de-energized, the plug 70 may close an opening 72 (shown in FIG. 9) in the vent tube 62. The plug's 70 soft material may help render silent the opening and closing of the vent tube opening 72. In alternative embodiments, the opening 72 in the vent tube 62 may be any size and may be located in the coaxial side walls of the vent tube 62 or in the end wall. The plunger 68 may be made of any magnetic material and may be a filament or wire in one embodiment. The plunger 68 may keep the plug 70 centered over the opening 72. In another embodiment, the plunger 68 may also be coated with a soft material. In some embodiments, the plug 70 or plunger 68 may be made from or coated with lightweight material. In an alternative embodiment, the vent solenoid 64 may comprise a ball bearing (made of magnetic material, coated with soft material) instead of a plunger 68 and plug 70, where the ball bearing may close the vent tube opening 72 similar to the plug 70.

FIG. 9 shows a partial cross-sectional view of the vent tube 62 and vent solenoid 64 in the energized and open position, according to one embodiment. When the coil 66 is energized, the plunger 68 may extend upwards (into the solenoid or the area surrounded by its coil), removing the plug 70 from the opening 72 and venting the vent tube 62. The diameter of the plug 70 and opening 72 may be less than the internal diameter of the vent tube 62 to allow air to get around the plug 70 to create the leak.

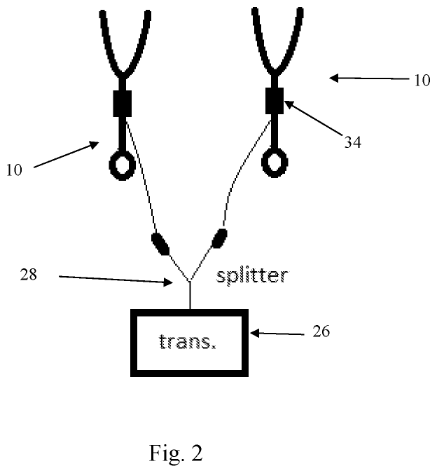

According to another embodiment, the training device 10 may include an enclosure 34 through which the tubing 18 passes, as shown in FIG. 2. The tubing 18 extending from the Y fork to the headpiece 12 may be separated into two lengths or pieces, with the enclosure 34 inserted between the two lengths such that a portion of each length passes through an opening in the enclosure 34. The enclosure 34 may house at least one of the following components: speaker 20, solenoid 22, audio jack 24, wireless receiver (discussed below), output 40, LED 42, controller 44, opening 60, vent tube 62, and vent solenoid 64.

According to one embodiment, the speaker 20 can be connected to a wireless receiver, including by way of the 3.5 mm audio jack 24. The wireless receiver can be configured to receive a wireless signal that has been broadcasted from a separate device and relay the wireless signal to the speaker 20 as an audio file for playback to the student. The wireless receiver can be configured to communicate via any suitable wireless technology, including, but not limited to, Wi-Fi, Bluetooth, radio frequency (RF), infrared (IR), ZigBee, etc.

With reference to FIG. 2, the external audio output device can consist of a transmitter 26 and a splitter 28 which can allow two of the training devices 10 to be connected simultaneously. According to one embodiment, two students can connect a training device 10 to the same audio output device allowing each student to hear the same sounds at the same time.

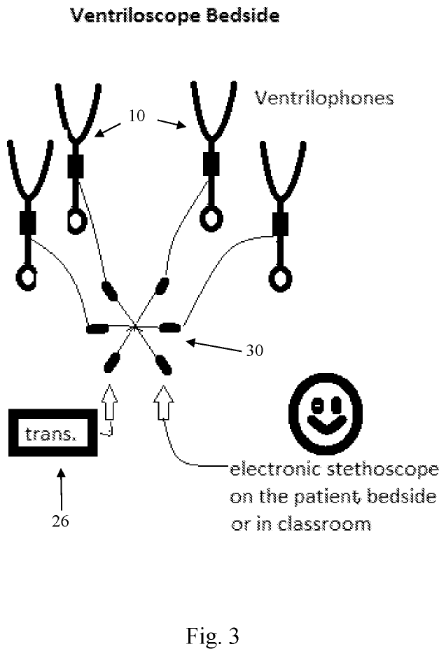

With reference to FIG. 3, the splitter 28 can be a multi-way splitter 30 having two or more connections. According to the embodiment shown, the multi-way splitter 30 can have six connections allowing up to five training devices 10 to connect to a single audio output device. According to one embodiment, the multi-way splitter 30 can be connected to four training devices 10, an audio output device, and an electronic stethoscope and transmitter, such as the Ventriloscope®, trademarked and owned by the present applicant and the subject matter of the '141 patent referenced herein. According to this embodiment, the training devices 10 can receive signals from either the audio output device, the electronic stethoscope, or both. This arrangement can allow multiple students to simultaneously hear sounds generated from a patient that is located remote from the students, such as in a bed in another room or in a classroom.

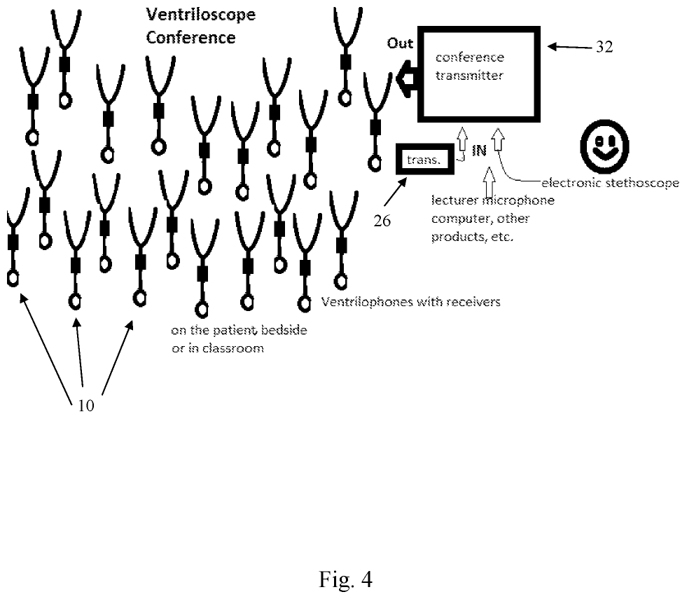

With reference to FIG. 4, a multitude of training devices 10 can each be connected to a wireless receiver via the 3.5 mm audio jack 24. The external audio output device can be connected to a remote transmitter 32 that is capable of receiving an audio signal from the audio device and broadcasting the signal to the multitude of training devices 10 via wireless technology. According to one embodiment, the wireless technology can be RF transmission. The remote transmitter 32 can also accept input from a lecturer, a microphone, a computer, or any other input capable of generating an audio signal. The remote transmitter 32 can also be connected to an electronic stethoscope for transmission of simulated or live patient sounds.

According to one embodiment, a patient or instructor can control which sounds the students are hearing according to the placement of a stethoscope on a patient or training simulator.

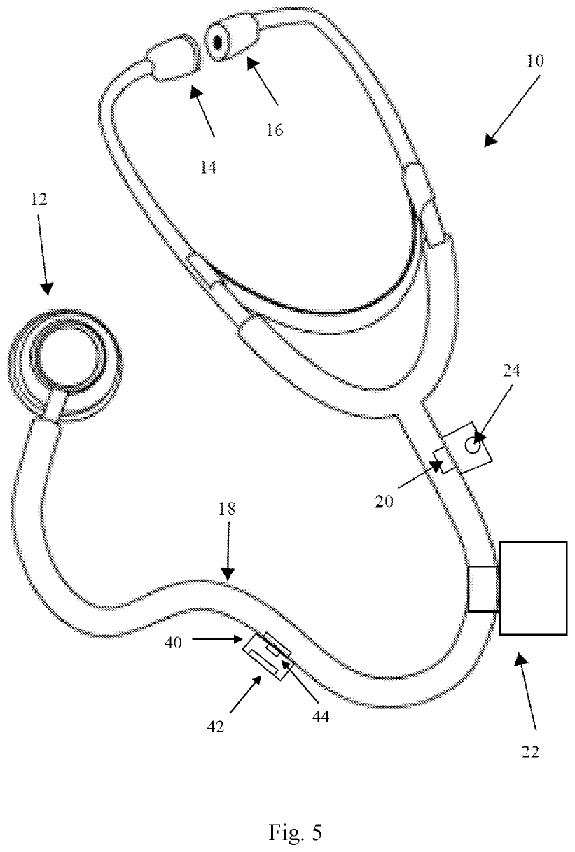

With reference to FIG. 5, the training device 10 can include an output 40, thereby allowing the training device 10 to broadcast or record live body sounds to other training devices 10, to an external storage device such as a computer or memory card, to an external audio device, or to any other device capable of receiving and or storing an audio signal. The output 40 can be integrated into the speaker 20 or can be a separate component disposed in line with the speaker 20 and the headpiece 12. The output 40 can consist of a microphone, a recording device, a transmitter, or an audio output jack, or a combination thereof. This embodiment would allow for a training device 10 to be used as the external audio output device for other training devices 10. This would allow students to share live body sounds in real time, or to store live body sounds for future playback.

According to one embodiment, an amplification device can be connected to the training device 10 to allow for amplification of live or simulated body sounds to assist the hearing impaired.

With continued reference to FIG. 5, a light, LED, or other visual signal can be connected and synchronized to the live or simulated body sounds. By way of one example, an LED 42 and controller 44 can be connected to the output 40 of the training device 10 and synchronized so that the LED 42 illuminates simultaneously with the pulse or breathing sounds being heard through the earpieces 14, 16. This embodiment can provide visual feedback to observers who are not connected to one of the training devices 10 and can be helpful in learning to diagnose conditions where the live or simulated body sounds correlate with the timing of the pulse or breathing sounds.

According to another embodiment, the light, LED, or other visual signal can be connected to a sensor separate from the training device 10 to detect the live or simulated body sounds. By way of one example, the sensor could be an EKG lead. Other methods of detecting live or simulated body sounds, such as changing impedance, resistance, or physical movement could also be used in conjunction with or separate from the training device 10.

According to another embodiment, the controller 44 can be integrated with an external control device for the opening 60 such that the controller 44 can separately, but simultaneously, control both the visual signal and the opening 60

Although described as a medical training device, the present invention can have applicability outside the medical field in any situation where teaching using a stethoscope or listening device is used. One such example could be in the automotive industry. Thus, while the present invention has been described in connection with various illustrative embodiments, it is to be understood that other similar embodiments may be used or modifications and additions may be made to the described embodiments for performing the same function disclosed herein without deviating therefrom. Further, all embodiments disclosed are not necessarily in the alternative, as various embodiments may be combined or subtracted to provide the desired characteristics. Variations can be made by one having ordinary skill in the art without departing from the spirit and scope hereof. Therefore, the present invention should not be limited to any single embodiment, but rather construed in breadth and scope in accordance with the recitations of the appended claims.

Having thus described the invention, it is now claimed:

I claim:

1. An auscultation training device comprising:
a headpiece;
at least one earpiece;
tubing having a hollow interior, connected between the headpiece and the at least one earpiece, and capable of transmitting sound from the headpiece to the at least one earpiece;
a speaker inserted into the hollow interior of the tubing through an insertion point that forms an airtight seal with the tubing, wherein the speaker does not fully obstruct the hollow interior of the tubing;
a vent tube having (a) a hollow interior, (b) a first end secured to the tubing such that the tubing hollow interior and vent tube hollow interior are in fluid communication, (c) a second end, and (d) an opening; and
a vent solenoid secured to the second end of the vent tube and capable of blocking and unblocking the vent tube opening.

2. The auscultation training device of claim 1, wherein:
the vent solenoid comprises a plunger having a plug;
the plug is larger than the vent tube opening but smaller than the diameter of the vent tube hollow interior; and
the plug is capable of blocking and unblocking the vent tube opening.

3. The auscultation training device of claim 2 further comprising an input source comprising an audio jack or a receiver, wherein the input source is electrically connected to the speaker, wherein the speaker is configured to play sound received by the input source.

4. The auscultation training device of claim 3, wherein the vent tube is secured to the tubing along the length of the tubing between the insertion point and the headpiece.

5. The auscultation training device of claim 4, wherein the vent solenoid further comprises a coil and is configured such that (a) when the coil is energized, the plunger recedes into the solenoid and the plug unblocks the vent tube opening, thereby venting the vent tube hollow interior and tubing hollow interior, and (b) when the coil is de-energized, the plunger extrudes from the solenoid and the plug blocks the vent tube opening.

6. The auscultation training device of claim 5, wherein the plug is made from or coated with a soft material selected from the group consisting of: foam, cotton, rubber, paper fiber, plastic, cloth, leather, and wool.

7. The auscultation training device of claim 6, wherein the plunger is configured to keep the plug centered over the vent tube opening.

8. The auscultation training device of claim 4, wherein:
the tubing comprises (a) a first portion, and (b) a second portion;
a first end of the tubing first portion is connected to the headpiece and a second end of the tubing first portion is connected to a first end of the tubing second portion; and
a second end of the tubing second portion is connected to the at least one earpiece.

9. The auscultation training device of claim 8 further comprising an enclosure that houses the speaker, the vent tube, the vent solenoid, the input source, and a part of the tubing first portion.

10. The auscultation training device of claim 9, wherein:
the tubing first portion comprises two pieces, and
the part of the tubing first portion that is housed inside the enclosure includes the location where the tubing first portion is separated into the two pieces.

11. The auscultation training device of claim 4 further comprising a blocking solenoid inserted into the hollow interior of the tubing through a second insertion point along the outer circumference and length of the tubing between the headpiece and the speaker, wherein the second insertion point of the blocking solenoid through the tubing forms an airtight seal with the tubing and wherein the blocking solenoid is selectively operational to fully obstruct the hollow interior of the tubing when activated.

12. The auscultation training device of claim 1, wherein the vent solenoid comprises a ball bearing that is:
larger than the vent tube opening but smaller than the diameter of the vent tube hollow interior;
capable of blocking and unblocking the vent tube opening; and
made from or coated with a soft material selected from the group consisting of: foam, cotton, rubber, paper fiber, plastic, cloth, leather, and wool.

13. The auscultation training device of claim 4, wherein the vent solenoid further comprises a coil and is configured such that (a) when the coil is de-energized, the plunger recedes into the solenoid and the plug unblocks the vent tube opening, thereby venting the vent tube hollow interior and tubing hollow interior, and (b) when the coil is energized, the plunger extrudes from the solenoid and the plug blocks the vent tube opening.

14. An auscultation training device comprising:
a headpiece;
at least one earpiece;
tubing having a hollow interior, connected between the headpiece and the at least one earpiece, and capable of transmitting sound from the headpiece to the at least one earpiece, wherein the tubing has a vent opening;
a speaker inserted into the hollow interior of the tubing through an insertion point that forms an airtight seal with the tubing, wherein the speaker does not fully obstruct the hollow interior of the tubing; and
a vent solenoid secured to the tubing and capable of blocking and unblocking the vent opening.

15. A method of auscultation training comprising the steps of:
(a) providing an auscultation training device comprising:
a headpiece;
at least one earpiece;
tubing having a hollow interior, connected between the headpiece and the at least one earpiece, and capable of transmitting sound from the headpiece to the at least one earpiece;
a speaker inserted into the hollow interior of the tubing through an insertion point that forms an airtight seal with the tubing, wherein the speaker does not fully obstruct the hollow interior of the tubing;
a vent tube having (1) a hollow interior, (2) a first end secured to the tubing such that the tubing hollow interior and vent tube hollow interior are in fluid communication, (3) a second end, and (4) an opening; and
a vent solenoid secured to the second end of the vent tube and capable of blocking and unblocking the vent tube opening;
(b) activating the vent solenoid to unblock the vent tube opening and vent the vent tube hollow interior and tubing hollow interior, dampening any sounds transmitted from the headpiece by the tubing to the at least one earpiece;
(c) playing a sound by the speaker; and
(d) transmitting the sound played by the speaker by the tubing to the at least one earpiece.

16. The method of claim 15 further comprising step:
(e) deactivating the vent solenoid and blocking the vent tube opening, allowing sounds to be transmitted from the headpiece by the tubing to the at least one earpiece without the dampening of step (b).

17. The method of claim 16, wherein:
the vent solenoid comprises a coil and a plunger having a plug capable of blocking and unblocking the vent tube opening;
the activating and unblocking of step (b) comprises:
energizing the coil;
the plunger receding into the solenoid; and
the plug unblocking the vent tube opening; and
the deactivating and blocking of step (e) comprises:
de-energizing the coil;
the plunger extruding from the solenoid; and
the plug blocking the vent tube opening.

18. The method of claim 17, wherein:
the auscultation training device further comprises an input source comprising an audio jack or a receiver;
the input source is electrically connected to the speaker; and
the sound played in step (c) is provided by the input source.

19. The method of claim 18,
wherein step (a) further comprises providing a remote transmitter;
the method further comprising step:
(f) wirelessly transmitting an audio signal from the remote transmitter to the receiver;
wherein the sound played in step (c) is from the audio signal received by the receiver.

20. The method of claim 18,
wherein step (a) further comprises providing an audio output device;
the method further comprising steps:
(g) connecting the audio output device to the audio jack; and
(h) transmitting an audio signal from the audio output device to the audio jack;
wherein the sound played in step (c) is from the audio signal received by the audio jack.

21. The method of claim 18, wherein step (a) further comprises providing a blocking solenoid inserted into the hollow interior of the tubing through a second insertion point along the outer circumference and length of the tubing between the headpiece and the speaker, wherein the second insertion point of the blocking solenoid through the tubing forms an airtight seal with the tubing and wherein the blocking solenoid is selectively operational to fully obstruct the hollow interior of the tubing when activated;
the method further comprising step:
(i) activating the blocking solenoid to obstruct the tubing hollow interior, dampening any sounds transmitted from the headpiece by the tubing to the at least one earpiece.

22. The method of claim 18, wherein step (a) further comprises providing an output;
the method further comprising step:
(j) transmitting through the output an audio signal representing sounds detected by the headpiece.

23. The method of claim 16, wherein:
the vent solenoid comprises a coil and a plunger having a plug capable of blocking and unblocking the vent tube opening;
the activating and unblocking of step (b) comprises:
de-energizing the coil;
the plunger receding into the solenoid; and
the plug unblocking the vent tube opening; and
the deactivating and blocking of step (e) comprises:
energizing the coil;
the plunger extruding from the solenoid; and
the plug blocking the vent tube opening.
